(19) United States
(12) Patent Application Publication
(10) Pub. No.: US 2026/0345901 A1
(43) Pub. Date: Nov. 5, 2026

(54) DEVICES, SYSTEMS, AND METHODS FOR IMPROVING SYRINGE PUMP OCCLUSION DETECTION AND FLOW CONTINUITY

(71) Applicant: CareFusion 303, Inc., San Diego, CA (US)

(72) Inventor: Vinay JANI, San Diego, CA (US)

(21) Appl. No.: 19/099,998

(22) PCT Filed: Aug. 3, 2023
(86) PCT No.: PCT/US2023/029438

§ 371 Date: Jan. 30, 2025

Related U.S. Application Data
(60) Provisional application No. 63/394,937, filed on Aug. 3, 2022.

(51) Int. Cl. A61M 5/168; A61M 5/145

(52) U.S. Cl. CPC A61M 5/16863; A61M 5/14546; A61M 2205/18; A61M 2205/332; A61M 2205/50

(57) ABSTRACT

A syringe pump includes a receptacle for receiving a syringe and a drive head for advancing the plunger of the syringe within the syringe's barrel. The syringe pump also includes a processor. The processor is configured to receive a programmed flow rate. The processor is also configured to detect an extension of the syringe, the extension including a distance between a starting position of the plunger within the barrel and a current position of the plunger within the barrel. Additionally, the processor is configured to estimate an amount of friction between the plunger and the barrel based on the programmed flow rate and the detected extension. Further, based on the estimated amount of friction, the processor is configured to adjust an occlusion-detection operation of the syringe pump or adjust an operating speed of the drive head.

### REFERENCE TO RELATED APPLICATIONS

This application claims priority to U.S. Provisional Patent Application 63/394,937, filed Aug. 3, 2023, the entire disclosure of which incorporated herein by reference.

### TECHNICAL FIELD

The present disclosure relates generally to syringe pumps. In particular, the present disclosure relates to devices, systems, and methods for improving syringe pump occlusion detection and flow continuity.

### BACKGROUND

In the medical field, syringe pumps are used regularly to provide patients with medications and other fluids. Currently, many syringe pumps include a force sensor for determining the amount of force exerted by the syringe plunger on the drive head of the syringe pump. This information can be used to detect downstream occlusions in the tubing that connects the syringe to the patient. However, this method of occlusion detection can be at times inaccurate, resulting in false-positive alarms.

Errant alarms pull clinicians away from their patients and contribute to alarm fatigue. Furthermore, errant alarms can consume limited resources of the infusion device, such as memory, user interface space, processor resources, network resources, and the like that may be needed for authentic infusion operations. Accordingly, there is a need for syringe pumps capable of more accurate occlusion detection. Furthermore, there is also a need for improving the flow continuity and flow rate accuracy of syringe pumps to ensure that patients receive their fluids at prescribed rates.

### SUMMARY

The present disclosure provides devices, systems, and methods for improving the occlusion detection and flow continuity of syringe pumps. Example implementations of the advancements discussed herein include the following:

A syringe pump includes a receptacle for receiving a syringe that includes a plunger and a barrel. The syringe pump also includes a drive head for advancing the plunger within the barrel. Additionally, the syringe pump includes a processor for performing various operations. The operations include receiving a programmed flow rate. The operations also include detecting an extension of the syringe, including a distance between a starting position of the plunger within the barrel and a current position of the plunger within the barrel. Additionally, the operations include estimating, based on the programmed flow rate and the detected extension, an amount of friction between the plunger and the barrel. Moreover, the operations include adjusting, based on the estimated amount of friction, an occlusion-detection operation of the syringe pump or an operating speed of the drive head.

A computer-implemented method for improving occlusion detection or flow continuity of a syringe pump includes receiving a programmed flow rate for a syringe pump. The syringe pump includes a receptacle for receiving a syringe that includes a plunger and a barrel. The syringe pump also includes a drive head for advancing the plunger within the barrel. The computer-implemented method also includes detecting an extension of the syringe, including a distance between a starting position of the plunger within the barrel and a current position of the plunger within the barrel. Additionally, the computer-implemented method includes estimating, based on the programmed flow rate and the detected extension, an amount of friction between the plunger and the barrel. Moreover, the computer-implemented method includes adjusting, based on the estimated amount of friction, an occlusion-detection operation of the syringe pump or an operating speed of the drive head.

A non-transitory, computer-readable storage medium comprising instructions that, when executed by an electronic device, cause the electronic device to perform various operations. The operations include receiving a programmed flow rate for a syringe pump. The syringe pump includes a receptacle for receiving a syringe that includes a plunger and a barrel. The syringe pump also includes a drive head for advancing the plunger within the barrel. The operations also include detecting an extension of the syringe, including a distance between a starting position of the plunger within the barrel and a current position of the plunger within the barrel. Additionally, the operations include estimating, based on the programmed flow rate and the detected extension, an amount of friction between the plunger and the barrel. Moreover, the operations include adjusting, based on the estimated amount of friction, an occlusion-detection operation of the syringe pump or an operating speed of the drive head.

Although the present disclosure describes the subject technology in the context of syringe pumps, much of the subject technology is nonetheless applicable to other types of infusion pumps. It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Detailed Description, below, in conjunction with the following drawings. Like reference numerals refer to corresponding parts throughout the figures and description.

FIG. 1 depicts an example infusion device including a control module, a peristaltic infusion pump, and a syringe pump, according to various aspects of the subject technology.

FIG. 2 depicts an example institutional patient care system of a healthcare organization, according to various aspects of the subject technology.

FIG. 3 depicts an example syringe pump module, according to various aspects of the subject technology.

FIG. 4A-4C depict an example standalone syringe pump, as well as example positions of a syringe received thereby, according to various aspects of the subject technology.

FIG. 5 depicts example forces acting on a drive head and a syringe, according to various aspects of the subject technology.

FIG. 6 is an example line graph that includes a friction-estimation curve and corresponding syringe pump adjustments, according to various aspects of the subject technology.

FIG. 7 depicts an example process for improving occlusion detection or flow continuity of a syringe pump, according to various aspects of the subject technology.

FIG. 8 is a conceptual diagram illustrating an example electronic system for improving occlusion detection or flow continuity of a syringe pump, according to various aspects of the subject technology.

### DETAILED DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Many modern syringe pumps use force sensors for downstream occlusion detection. The force sensors detect the amount of force needed to compress the syringe plunger, which force can then be translated into an estimate of the amount of pressure in the syringe. For example, some syringe pumps employ a force sensor situated between the drive head and the syringe plunger to detect an amount of force applied to the drive head by the plunger. This process generally relies on the assumption that the detected force is roughly equal to the amount of force experienced by the fluid in the syringe.

However, this assumption can sometimes be false. For example, friction between the plunger and the barrel of the syringe (e.g., resisting lateral movement of the plunger within the barrel) can cause the force experienced by the fluid within the syringe to deviate from (e.g., be less than) the amount of force needed to compress the syringe plunger. This in turn can lead to improper occlusion alarms due to pressure calculations based on incomplete force data, as discussed in more detail below. Moreover, friction experienced by the plunger can also disrupt the flow rate continuity of an infusion therapy, causing inconsistent advancement of the drive head and the syringe plunger. This is especially common at the start of an infusion therapy, where friction between plunger and barrel may be at its greatest.

Accordingly, the present disclosure provides a refined approach to occlusion detection and syringe pump operation by estimating and accounting for forces, like friction, that might otherwise lead to false alarms and/or sub-par pump operation. Decreasing errant occlusion alarms can help to preserve pump resources and increase the amount of time clinicians are able to spend with their patients. Further, improving flow rate continuity and flow rate accuracy can improve patient treatment by ensuring that patients receive prescribed fluids at proper rates. Moreover, compensating for friction can also help to reduce or eliminate startup delay in an infusion therapy.

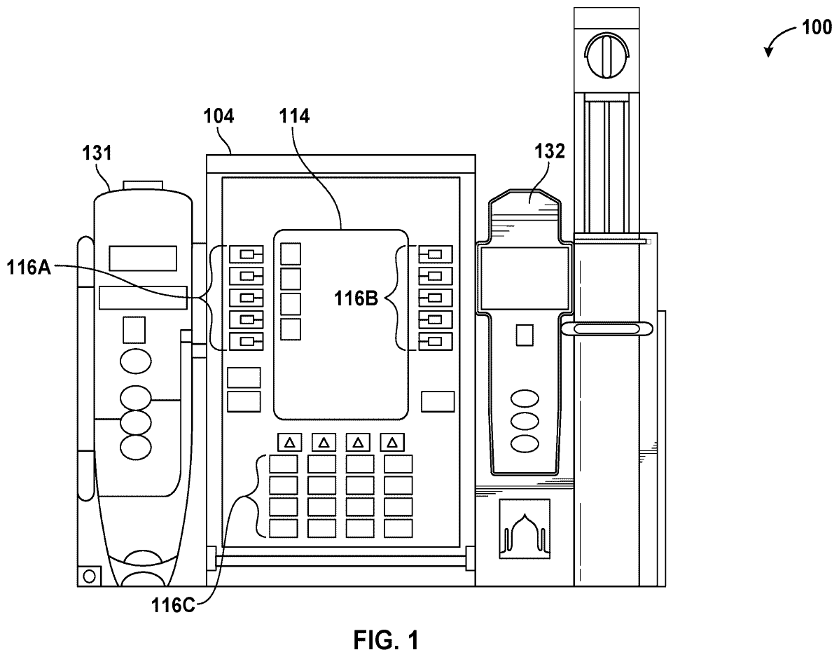

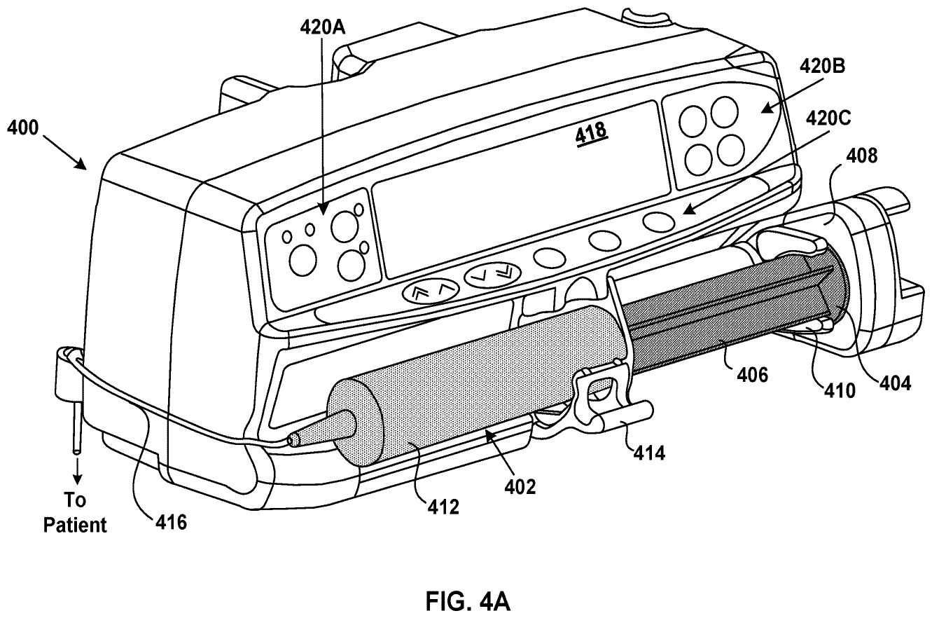

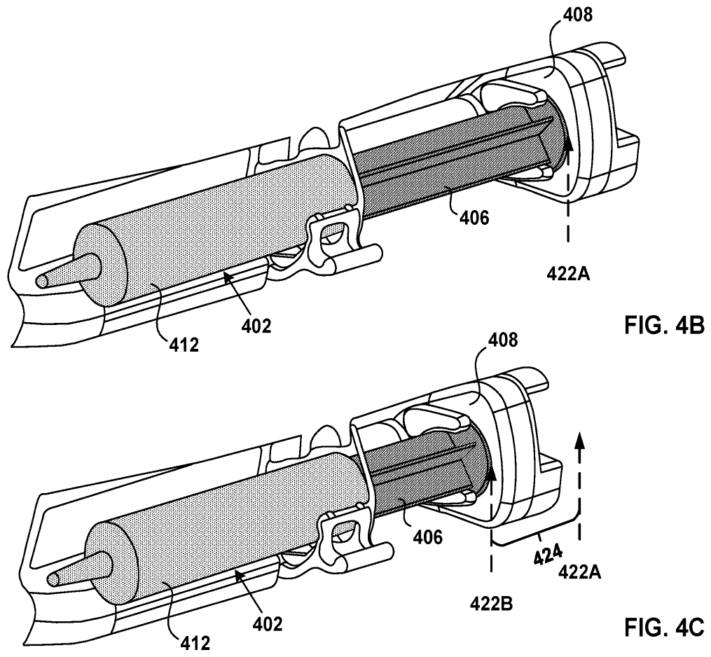

The first four figures, FIGS. 1-4, discussed immediately below, provide a foundation for discussing improved occlusion detection and flow continuity. They depict example infusion devices (e.g., syringe pumps) and illustrate how the devices might be used in medical contexts. Starting with FIG. 4, the present disclosure discusses metrics relevant to friction estimations, such as the extension of the syringe plunger within the barrel and the programmed flow rate of the syringe pump. This discussion continues with the remaining figures, which further flesh out devices, systems, and methods for improving the operations of syringe pumps.

FIG. 1 depicts an example infusion device 100 including a control module 104, a peristaltic infusion pump 131, and a syringe pump 132, according to various aspects of the subject technology. As depicted, the peristaltic infusion pump 131 and the syringe pump 132 (collectively, infusion pumps 131 and 132) are mounted at either side of the control module 104, which is configured for programming the infusion pumps 131 and 132.

According to various implementations, the control module 104 is used to provide a user interface for the infusion pumps 131 and 132. The control module 104 may also act as an interface between the infusion pumps 131 and 132 and external devices (e.g., a device terminal, a smartphone or tablet computer). As depicted in FIG. 1, the control module 104 includes a display 114 for visually presenting various information to a clinician, such as operating parameters of the infusion pumps 131 and 132 or alerts (e.g., alert indications, alert messages) relating to the infusion pumps 131 and 132. The control module 104 may also include a speaker to provide audible alerts. In some implementations, the display 114 may be implemented as a touchscreen display. In this manner, the control keys 116A-C may be omitted or reduced in number by providing corresponding interactive elements via a graphical user interface presented via the display 114. In some implementations, the infusion pumps 131 and 132 may include hardware elements to provide a human perceivable manifestation of an alert or alarm (e.g., regarding an occlusion) to a clinician such as a light (e.g., a variable-color light emitting diode), a display, or an audio output (e.g., speaker).

Additionally, the control module 104 may include a communications system by which the control module 104 may communicate with external equipment. For example, the control module 104 may communicate with a medical facility server, a computer (e.g., a handheld communication device or a laptop-type of computer), or an information device. In communicating with these devices, the control module 104 may transfer information to the devices, or the control module 104 may download information (e.g., drug libraries) from them.

The communications system may be used to transfer access and interaction information for users encountering the control module 104 or a device coupled therewith (e.g., infusion pumps 131 and 132, or a bar code scanner). Additionally, the communications system may include one or more of a radio frequency (RF) system, an optical system such as infrared, a BLUETOOTH™ system, or other wired or wireless system. A communications system (and/or the aforenoted barcode scanner) may additionally be included integrally with the infusion pumps 131 and 132, such as in implementations where the infusion device 100 does not include a control module. Information input devices need not be hard-wired to medical instruments; information may be transferred through a wireless connection, as well.

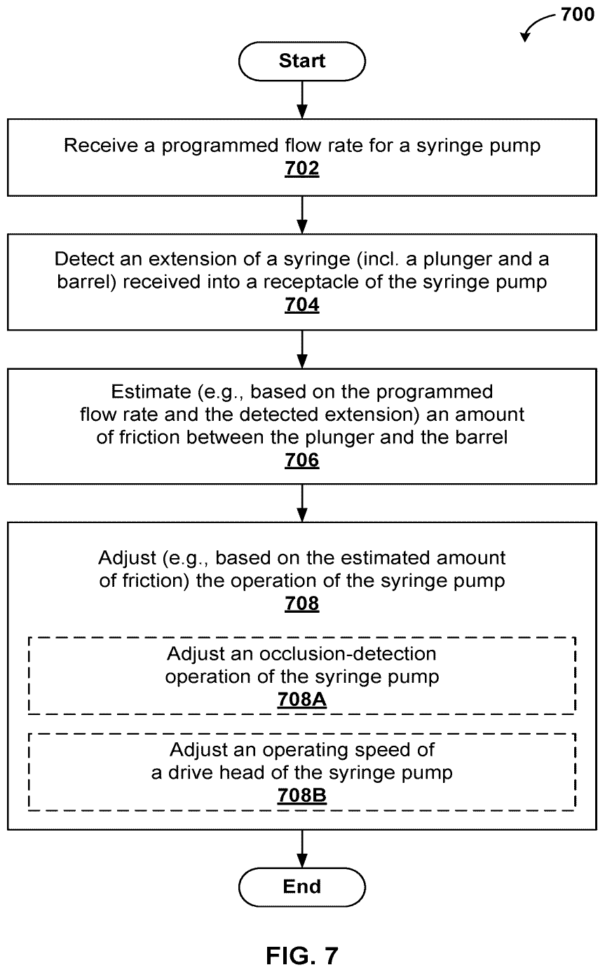

In addition to the infusion pumps 131 and 132, the control module 104 may also be connected to other functional modules, such as physiological monitors (e.g., heart rate, blood pressure, ECG, EEG, or pulse oximeter monitors), therapy devices, or other drug delivery devices (e.g., additional infusion pumps), according to the teachings set forth herein. Moreover, the control module 104 may a central processing unit (CPU) connected to a memory, such as random access memory (RAM). In some implementations, the control module 104 includes a main, non-volatile storage unit, such as a hard-disk drive or a non-volatile flash memory. For example, the control module 104 may for store software data on the non-volatile storage unit. Additionally, the control module may include one or more internal buses for connecting the aforementioned elements (see FIG. 7).

In various implementations, the display 114 is a touch screen for displaying information to a user and allowing a user to input information by touching defined areas of the screen. Additionally, or in the alternative, the display 114 could include any means for displaying and inputting information, such as a monitor, a printer, a keyboard, softkeys, a mouse, a track ball, and/or a light pen.

The control module 104 may include a data input device, such as a bar code reader capable of scanning and interpreting data printed in bar-coded format. Additionally, or in the alternative, the data input device can be a device for entering coded data into a computer, such as a device(s) for reading magnetic strips, radio-frequency identification (RFID) devices whereby digital data encoded in RFID tags or smart labels are captured by the data input device via radio waves, PCMCIA smart cards, radio frequency cards, memory sticks, CDs, DVDs, or any other analog or digital storage media. Other examples of the data input device include a voice activation or recognition device or a portable personal data assistant (PDA).

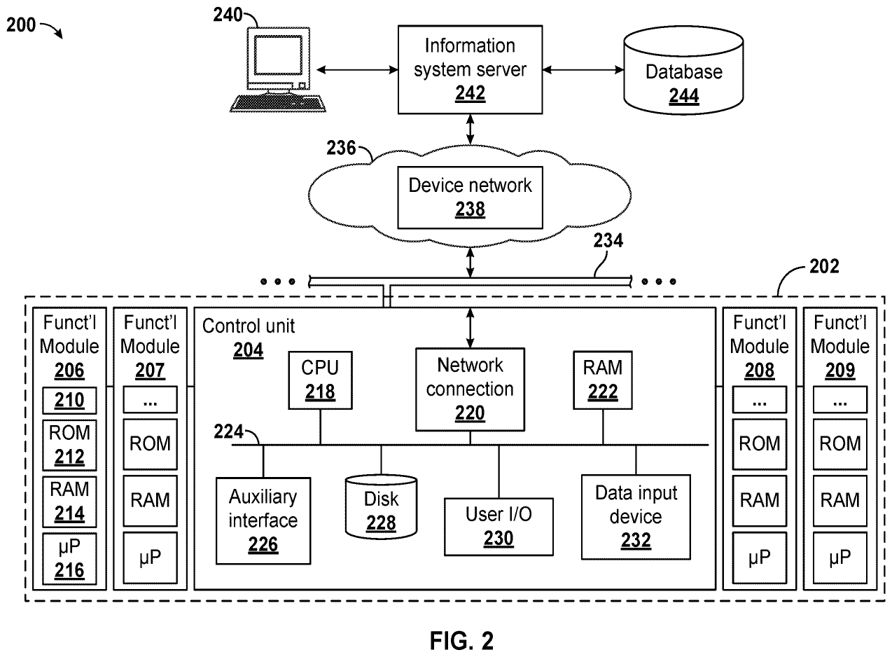

FIG. 2 depicts an example institutional patient care system 200 of a healthcare organization, according to various aspects of the subject technology. In FIG. 2, a patient care device 202 (e.g., infusion device 100 of FIG. 1) is connected to an internal healthcare network 236. The term patient care device, or “PCD,” may be used interchangeably with the term patient care unit, or “PCU,” either of which may include various ancillary medical devices such as an infusion pump (e.g., infusion pumps 131 and 132 of FIG. 1), a vital signs monitor, a medication dispensing device (e.g., cabinet, tote), a medication preparation device, an automated dispensing device, a module coupled with one of the aforementioned (e.g., a syringe pump module coupled with an infusion pump), or other similar devices. Each element of the patient care device 202 is connected to an internal healthcare network 236 by a transmission channel 234. Transmission channel 234 is any wired or wireless transmission channel, such as an 802.11 wireless local area network (LAN).

In some implementations, the internal healthcare network 236 also includes computer systems located in various departments throughout a hospital. For example, the network 236 optionally includes computer systems associated with an admissions department, a billing department, a biomedical engineering department, a clinical laboratory, a central supply department, one or more unit station computers, and/or a medical decision support system. As described further below, the internal healthcare network 236 may include discrete subnetworks. In the depicted example, the internal healthcare network 236 includes a device network 238 by which the patient care device 202 and other devices communicate in accordance with normal operations.

Additionally, institutional patient care system 200 may incorporate a separate information system server 242. Moreover, although the information system server 242 is shown as a separate server, the functions and programming of the information system server 242 may be incorporated into another computer. Institutional patient care system 200 may further include a device terminal 240 for connecting and communicating with information system server 242. The device terminal 240 may include personal computers, personal data assistances, and mobile devices such as laptops, tablet computers, augmented reality devices, or smartphones, configured with software for communications with information system server 242 via the internal healthcare network 236.

Patient care device 202 comprises a system for providing patient care, and may include or incorporate pumps (e.g., infusion pumps 131 and 132 of FIG. 1), physiological monitors (e.g., heart rate, blood pressure, ECG, EEG, pulse oximeter, and other patient monitors), therapy devices, and other drug delivery devices may be utilized according to the teachings set forth herein.

In the depicted example, patient care device 202 comprises a control module 204 (e.g., control module 104 of FIG. 1) connected to one or more functional modules 206-209 (e.g., infusion pumps 131 and 132 of FIG. 1). Control module 204 includes a central processing unit (CPU) 218 connected to a memory, for example, random access memory (RAM) 222, and one or more interface devices such as user interface device 230, a coded data input device 232, a network connection 220, and an auxiliary interface 226 for communicating with additional modules or devices. Control module 204 also, although not necessarily, includes a main non-volatile storage unit 228, such as a hard disk drive or non-volatile flash memory, for storing software data. Additionally, control module 204 may include one or more internal buses 224 for interconnecting the aforementioned elements.

In various implementations, user interface device 230 is a touch screen for displaying information to a user and allowing a user to input information by touching defined areas of the screen. Additionally, or in the alternative, user interface device 230 could include means for displaying and inputting information, such as a monitor, a printer, a keyboard, softkeys, a mouse, a track ball, and/or a light pen.

Data input device 232 may be a bar code reader capable of scanning and interpreting data printed in bar coded format. Additionally, or in the alternative, data input device 232 can be any device for entering coded data into a computer, such as a device(s) for reading magnetic strips, radio-frequency identification (RFID) devices whereby digital data encoded in RFID tags or smart labels (defined below) are captured by the data input device 232 via radio waves, PCMCIA smart cards, radio frequency cards, memory sticks, CDs, DVDs, or any other analog or digital storage media. Other examples of the data input device 232 include a voice activation or recognition device or a portable personal data assistant (PDA).

Depending upon the types of interface devices used, the user interface device 230 and the data input device 232 may be the same device. Although the data input device 232 is shown in FIG. 2 as being disposed within the control module 204, it is recognized that the data input device 232 may be external to the control module 204 (e.g., at the device terminal 240).

Auxiliary interface 226 may be an RS-232 communications interface, however any other means for communicating with a peripheral device (e.g., a printer, a patient monitor, an infusion pump, or another medical device) may be used without departing from the subject technology. Additionally, the data input device 232 may be a separate functional module (e.g., one of functional modules 206-209) configured to communicate with the control module 204 or any other system on the network using suitable programming and communication protocols.

Network connection 220 may be a wired or wireless connection, such as by Ethernet, WiFi, BLUETOOTH, an integrated services digital network (ISDN) connection, a digital subscriber line (DSL) modem or a cable modem. Any direct or indirect network connection may be used, including, but not limited to a telephone modem, an MIB system, an RS232 interface, an auxiliary interface, an optical link, an infrared link, a radio frequency link, a microwave link or a WLANS connection or other wireless connection.

The functional modules 206-209 are devices (e.g., infusion pumps 131 and 132 of FIG. 1) for providing care to a patient or for monitoring patient conditions. As shown in FIG. 2, at least one of functional modules 206-209 may be an infusion pump module such as a syringe pump (e.g., syringe pump 132 of FIG. 1) for delivering medication or other fluid to a patient. For the purposes of this discussion, functional module 206 is referred to as an infusion pump module. However, it is noted that each of functional modules 206-209 may be any patient treatment or monitoring device including, but not limited to, an infusion pump, a syringe pump, a PCA pump, an epidural pump, an enteral pump, a blood pressure monitor, a pulse oximeter, an EKG monitor, an EEG monitor, a heart rate monitor, an intracranial pressure monitor, or the like. Additionally, the functional modules 206-209 may also be a printer, a scanner, a bar code reader, a near-field communication reader, an RFID reader, or any other peripheral input, output or input/output device.

Each functional module 206-209 communicates directly or indirectly with control module 204, providing overall monitoring and control of the patient care device 202. Additionally, the functional modules 206-209 may be connected physically and electronically in serial fashion to one or both ends of control module 204 as shown in FIG. 2.

It is recognized that there are other means for connecting the functional modules 206-209 with the control module 204 that may be utilized without departing from the subject technology. It is also appreciated that devices such as pumps or patient monitoring devices that provide sufficient programmability and connectivity may be capable of operating as stand-alone devices and may communicate directly with the internal healthcare network 236 without being connected through the control module 204 or a separate interface unit. As described above, additional medical devices or peripheral devices may be connected to the patient care device 202 through one or more auxiliary interfaces 226.

Each of the functional modules 206-209 may include a microprocessor 216, a volatile memory 214, a nonvolatile memory 212, and module-specific components 210. It should be noted that while four functional modules are shown in FIG. 2, any number of devices may be connected directly or indirectly to the control module 204. The number and type of functional modules described herein are intended to be illustrative, and they in no way limit the scope of the subject technology. The module-specific components 210 include any components necessary for operation of a particular module, such as a pumping mechanism for the functional module 206.

While each of the functional modules 206-209 may be capable of a least some level of independent operation, the control module 204 monitors and controls overall operation of the patient care device 202. For example, as will be described in more detail below, the control module 204 provides programming instructions to the functional modules 206-209 and monitors the status of each of the functional modules 206-209.

Medical devices incorporating aspects of the subject technology may be equipped with a network interface module (NIM), allowing the medical device to participate as a node in a network. While for purposes of clarity the subject technology will be described as operating in an Ethernet network environment using the Internet Protocol (IP), it is understood that concepts of the subject technology are equally applicable in other network environments (e.g., standards-based or proprietary), and such environments are intended to be within the scope of the subject technology.

Data to and from the various data sources can be converted into network-compatible data with existing technology, and movement of the information between the medical device and network can be accomplished by a variety of means. For example, the patient care device 202 and the internal healthcare network 236 may communicate via automated interaction, manual interaction, or a combination of both automated and manual interaction. Automated interaction may be continuous or intermittent and may occur through direct network connection 220, as shown in FIG. 2, or through RS232 links, MIB systems, RF links such as BLUETOOTH, IR links, WLANS, digital cable systems, telephone modems, or other wired or wireless communication means.

Manual interaction between the patient care device 202 and the internal healthcare network 236 involves physically transferring, intermittently or periodically, data between systems using, for example, the user interface device 230, the coded data input device 232, bar codes, computer disks, portable data assistants, memory cards, or any other media for storing data. The communication means in various aspects is bidirectional with access to data from as many points of the distributed data sources as possible. Decision-making can occur at a variety of places within the internal healthcare network 236. For example, and not by way of limitation, decisions can be made in the information system server 242, decision support, a remote data server, hospital department or unit stations, or within the patient care device 202 itself.

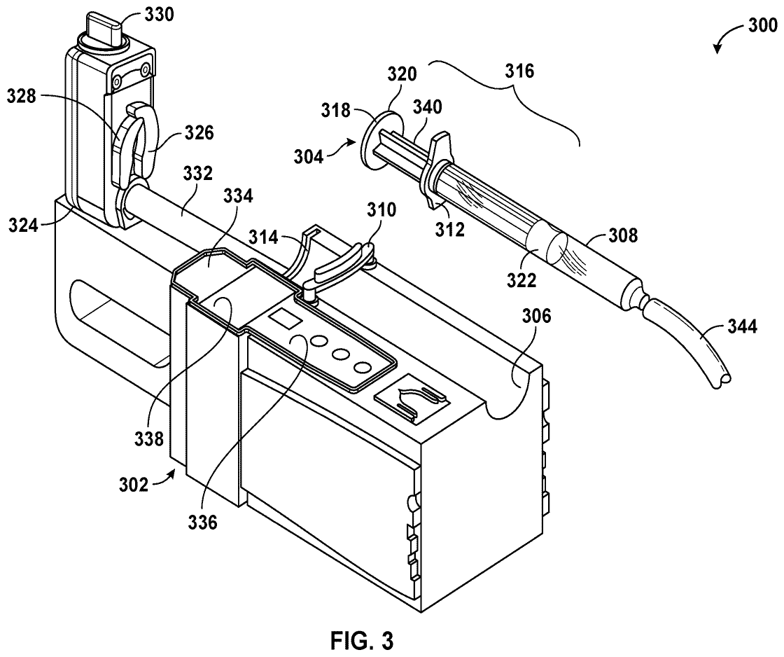

FIG. 3 depicts an example syringe pump module 302 (e.g., syringe pump 132 of FIG. 1), according to various aspects of the subject technology. In the depicted implementation, an example infusion system 300 includes the syringe pump module 302, which may include a drivetrain subsystem. A syringe 304 is shown next to the pump rather than mounted in the pump, for clarity of illustration. The syringe pump module 302 includes a cradle 306 in which a barrel 308 of the syringe 304 can rest when mounted in the syringe pump module 302. The cradle 306 can include a clamp 310 to securely hold the barrel 308 in a fixed position in the cradle 306 so that axial and lateral movement is resisted.

The clamp 310 can be pivoted so that it may be moved into an open position to permit loading or removal of the syringe 304 and a closed position in which it extends over the cradle 306 to hold a mounted barrel 308. A barrel flange 312 of the syringe 304 can be located in a barrel flange groove 314 in the syringe pump module 302 to immobilize the barrel 308 from axial movement during movement of the plunger 316 within the barrel 308.

In the example infusion system 300, the syringe 304 includes the barrel 308 and the plunger 316. The plunger 316 includes a push-button 318 having an inner side 320 and being interconnected with a stopper 322 of the plunger 316 by a piston 340. The plunger 316 can include the stopper 322 to engage an inner wall of the barrel 308 to prevent fluid from leaking past the stopper 322 (e.g., by creating a seal between the stopper 322 and the inner wall of the barrel 308).

The drive head 324 of the syringe pump module 302 may be connected to a screw-drive mechanism that includes a motor, for connecting the linear motion of the screw-drive mechanism to the plunger 316 in order to empty the contents of the syringe 304 through an administration set 344 and to a patient. The flow rate of the syringe pump module 302 can be controlled by the syringe pump module 302 based on programmed parameters (e.g., a requested flow rate, a type of the syringe).

When mounted in the syringe pump module 302, the push-button 318 can be held by the drive head 324 with a plunger retainer comprising a pair of pivotally mounted claws, first retainer claw 326 and second retainer claw 328, shown in the closed position in FIG. 3. The retainer claws 326 and 328 can curve inwardly toward each other to grasp the push-button 318 while the syringe 304 is mounted in the syringe pump module 302.

A rotation knob 330 can be used to control the positions of the first and second retainer claws 326 and 328 to allow removal and insertion of the push-button 318 and to release the split-nut from the driveshaft to permit axial positioning of the drive head 324. Also, the syringe 304 can be provided for use with the syringe pump module 302 with different quantities of fluid, such that the plunger 316 of the syringe 304 may be located at different positions in relation to the barrel 308.

The drive head 324 may be manually adjustable to accommodate syringes with different beginning plunger positions. A syringe inserted in the cradle 306 can align with the drive head 324 within a particular axial range. The points where the axial center lines of the syringes intersect the driver can change according to the size of the syringe but only in one direction along the drive head 324. A guide device 332 can extend from the drive head 324 to a point within a body of the syringe pump module 302.

The syringe pump module 302 can include a control panel 334 providing multiple buttons 336 for control of the syringe pump module 302 as well as a display 338 used to present pump-specific information to the operator. The buttons 336 can allow the operator to program the syringe pump module 302 for the flow rate, the volume to be infused, and/or other parameters. The display 338 can present the programmed flow rate, the amount of fluid remaining to be infused, as well as alarms and other information.

FIGS. 4A-4C depict an example standalone syringe pump 400, as well as example positions 422A and 422B of a syringe 402 received thereby, according to various aspects of the subject technology. While the example syringe pump 400 is shown as a standalone device, the syringe pump 400 may be configured as a functional module of a modular infusion system, such as a syringe module (e.g., syringe pump 132 of FIG. 1 or syringe pump module 302 of FIG. 3) in a patient care device (e.g., infusion device 100 of FIG. 1).

As noted above, friction between the plunger and the barrel of a syringe can lead to false-positive occlusion alarms, as well as inconsistent flow rates, particularly when detection of an occlusion alarm is based on force measured at the drive head. Determining the amount of friction between the plunger and barrel of a syringe can be difficult, however, because syringe stoppers (see, e.g., stopper 322 of FIG. 3) are often made of materials, such as rubber, which may have varying amounts of viscoelasticity (e.g., as also affected by the fluid in the syringe).

According to various implementations, friction can be accurately estimated based on one or more factors, including the extension of the plunger within the barrel. As used herein, “extension” or “syringe extension” (or “drive head extension”) refers to the amount of distance (e.g., in cm or mm) between a starting position of the syringe plunger (or the drive head) at the start of an infusion therapy, or after pausing an infusion therapy and allowing the syringe pump to come to a rest, and the current position of the syringe plunger (or the drive head). This is discussed in more detail below with respect to FIGS. 4B and 4C.

Starting first with FIG. 4A, similar to the implementations of FIG. 3, when the syringe 402 is loaded into the syringe pump 400, a plunger flange 404 at the end of a syringe plunger 406 is held in or against a drive head 408 by a flange clamp 410. The syringe barrel 412 is secured by a barrel clamp 414. The drive head 408 includes a pushing surface on which the plunger flange 404 will rest as the drive head 408 moves toward the syringe barrel 412, thus pushing the syringe plunger 406 into the secured syringe barrel 412 to expel the contents of the syringe 402 through an administrative set 416 and to the patient.

As with the drive head 324 of FIG. 3, the drive head 408 may be connected to a screw-drive mechanism that includes a motor, for connecting the linear motion of the screw-drive mechanism to the syringe plunger 406 in order to empty the syringe 402. The flow rate of the syringe pump 400 can be controlled by the syringe pump 400 based on programmed parameters (e.g., a requested flow rate, a type of the syringe).

Typically, syringe pumps do not experience upstream pressure conditions because the fluid to be infused is housed in the syringe barrel 412 and pushed into the administrative set 416 by way of the syringe plunger 406. Accordingly, in some implementations, the syringe pump 400 does not include an upstream pressure sensor. Downstream pressure conditions, on the other hand, are more commonly experienced by syringe pumps. Accordingly, in some implementations, the syringe pump 400 includes a force sensor (not shown) for detecting said downstream pressure. In some implementations, a force sensor measures the force exerted by the drive head 408 of the syringe pump on the syringe plunger 406.

In some implementations, the syringe pump 400 includes a high-resolution force sensor that interfaces with a pressure disc on the administrative set 416. The pressure disc provides a relatively large area in contact with the pressure sensor. This allows the sensor to measure the pressure inside the administrative set 416 more directly (e.g., rather than through the head of the syringe plunger 406) and with higher resolution and higher accuracy as compared to a force sensor located at the drive head 408. The measurements from the high-resolution force sensor and those from the drive head force sensor can be used independently or in conjunction with each other to detect an empty condition in the syringe pump 400.

In addition to various buttons or switches, which the operator may use to activate and program the syringe pump 400, there is also a display screen 418. The display screen 418 may be a liquid-crystal display (LCD) having a small number of segments, for example seven segments in a figure-of-eight configuration per character, adapted to display a small number of alphanumeric characters.

Additionally, the display 418 may be monochromatic. For example, it might only display red, green, or black characters. Alternatively, the display 418 can be a more complicated LCD capable of displaying more characters and/or more complicated characters.

The LCD may be backlit, for example, using light emitting diodes (LEDs). In some implementations, the infusion pump may include a thin-film transistor (TFT) LCD. In some implementations, the display 418 is a touchscreen, such as a capacitive touchscreen.

When programming the syringe pump 400, a user may input the type of the syringe 402 being used. The syringe pump 400 may store in an internal memory a database of known syringe types containing information such as syringe diameter and/or stroke. In this manner, the infusion pump firmware can calculate the position of the syringe plunger 406 based on movement of the drive head 408 and the type and size of the syringe 402. This may allow the machine to display the volume infused, time elapsed, volume remaining, and/or time remaining. As the infusion continues and the drive head moves, these calculations can be updated, and the information displayed at display 418 can be changed.

The syringe pump 400 may include an input interface with controls operable to enter, increase, and/or decrease pumping parameters (e.g., flow rate, or volume to be infused). As depicted, in some implementations, input keys 420A-C are physically present on the device. However, in some implementations, input keys are graphically displayed on the display 418 (e.g., a touchscreen display).

In some implementations, the syringe pump 400 may be configured to identify (e.g., using a sensor) a disposable container loaded by the device. For example, the syringe pump 400 may perform electro-mechanical measurements on the loaded syringe to identify certain characteristics about the loaded container. Additionally, in some implementations, the syringe pump 400 is configured to detect the size (e.g., diameter) of the syringe 402 inserted into the syringe pump 400. For example, the syringe pump 400 may include a sensor that measures the size of the syringe 402 (e.g., whether it is a 6 mL, a 10 mL, or a 50 ml syringe) based on how tightly the syringe is being hugged or based on the position of the barrel clamp 414. For example, based on measurements made by the sensor, the syringe pump may determine a list of possible candidate syringes. The device may then request confirmation via the display whether the container is within that list. During the infusion, volume infused and/or flow rate may be calculated based on the type of the syringe 402 (e.g., based on the size of the syringe barrel 412).

Turning now to FIGS. 4B and 4C, these two figures illustrate syringe extension-a key factor in determining the amount of friction between the plunger 406 and the barrel 412 of the syringe 402. In FIG. 4B, the plunger 406 is shown in its initial starting position (e.g., at the start of an infusion therapy), before being driven further into the barrel 412. The starting position of the plunger 406 is marked as position 422A. In FIG. 4C, the drive head 408 has pushed the plunger 406 forward into the barrel 412 such that the plunger 406 is now at position 422B. In this manner, the syringe extension is indicated by the bracket spanning distance 424, from position 422A to position 422B (e.g., the starting and current positions of the plunger, respectively).

In some implementations, the syringe pump 400 is configured to detect extension by recording a starting position of the drive head 408 and determining the current position of the drive head 408 based on the operating speed of the syringe pump (e.g., corresponding to the speed at which the drive head and the plunger are traveling) and the amount of time for which the syringe pump has been operating.

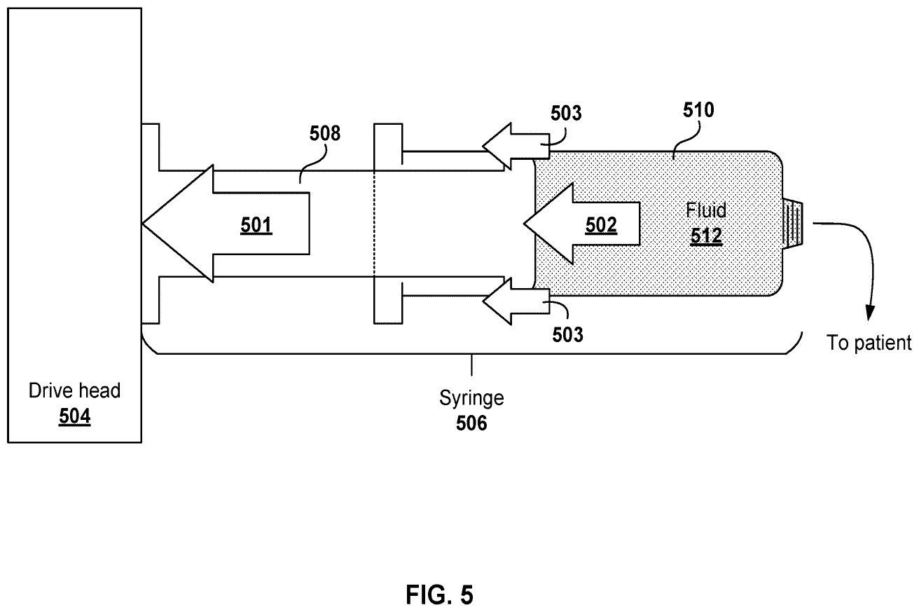

FIG. 5 depicts example forces 501-503 acting on a drive head 504 (e.g., drive head 324 of FIG. 3 or drive head 408 of FIGS. 4A-4C) and a syringe 506 (e.g., syringe 304 of FIG. 3 or syringe 402 of FIGS. 4A-4C), according to various aspects of the subject technology. A first force 501 is the result of the drive head 504 pressing on a plunger 508 (e.g., plunger 316 of FIG. 3 or plunger 406 of FIGS. 4A-4C) of the syringe 506. As the drive head 504 presses on the plunger 508, the plunger 508 travels into a barrel 510 (e.g., barrel 308 of FIG. 3 or barrel 412 of FIGS. 4A-4C) of the syringe 506 and presses a fluid 512 out of the syringe 506 and into a patient. This results in a second force 502: the force of the fluid 512 on the plunger 508. As discussed further below, the second force 502 may be contributory to the first force 501.

A syringe pump may approximate syringe pressure based on the assumption that the first 501 and second forces 502 are roughly equal. However, as illustrated in FIG. 5, a third force 503 may act on the plunger and prevent the entirety of the first force 501 from translating into the second force 502. This third force 503 is caused by friction between the plunger 508 and the barrel 510. Due to the elastoviscous nature of many plunger stoppers, the third force 503 can vary based on both the position of the plunger 508 relative to its starting position (e.g., syringe extension, see distance 424 of FIG. 4C) and the speed of the plunger 508 (e.g., corresponding to a programmed flow rate of the syringe pump and an associated operating speed of the drive head 504). This relationship between the three forces 501-503 can be represented mathematically, as follows:

F=P*A+D

In this formula, F is the first force 501, the force of the plunger 508 against the drive head 504. Next, P*A is the second force 502, the force of the fluid 512 against the syringe 506, split into component parts P and A, which represent, respectively, the pressure (P) in the barrel 510 and the surface area (A) of the bottom of the plunger 508. Finally, D is the third force 503, the friction between the plunger 508 and the barrel 510—resisting free movement of the plunger 508 against the barrel 510. Subtracting the third force 503 (D) from the first force 501 (F) results in the second force 502 (P*A). And dividing the result by the surface area (A) of the bottom of the plunger 508 yields the amount of pressure (P) in the syringe, as follows:

$$\hat{P} = \frac{F - \hat{D}}{A}$$

The amount of pressure in the syringe 506 (P) can then be used to determine whether an occlusion is present in or downstream from the syringe. For example, if the amount exceeds a pressure threshold, then there is likely an occlusion.

IF[\hat{P}>Pₐₗₐᵣₘ then ALARM, STOP, etc.]

In some implementations, the amount of friction between the plunger 508 and the barrel 510 is estimated using a machine learning (ML) model. The machine learning model may be trained, for example, using training data regarding measured amounts of friction at various degrees of syringe extension for a variety of syringes at different flow rates. In some implementations, the degree of syringe extension, the programmed flow rate (or corresponding operating speed), or a quality of the syringe (e.g., syringe capacity, syringe diameter, syringe manufacturer, and/or syringe model) may be controlled, such that the ML model is trained with data only for a particular degree of extension, a particular flow rate, and/or a particular syringe quality. For example, one ML model may be trained using data collected using only syringes with a capacity of 50 milliliters. Another ML model may be trained using data collected using only syringes from a particular manufacturer.

In some implementations, estimating the amount of friction between the plunger 508 and the barrel 510 includes providing parameters to an ML model (e.g., syringe extension and/or programmed flow rate) and then receiving the estimated amount of friction from the ML model. In some implementations, the ML model may be trained such that it need only receive a single parameter (e.g., syringe extension or programmed flow rate) in order to provide an estimated amount of friction between the plunger 508 and the barrel 510. A single-input model, in some instances, may be less computationally intensive than a multiple-input model. Accordingly, syringe pumps with less computational capacity may be restricted to using single-input models.

As an example, a multi-input ML model may be expressed in the form:

$$F = {w}_{1} {x}_{1} + {w}_{2} {x}_{2} + {w}_{3} {x}_{1} {x}_{2} + … + {w}_{n} {x}_{1}^{n} + {w}_{n + 1} {x}_{2}^{n} + {w}_{( n + 2 )} {x}_{1}^{m} {x}_{2}^{l}$$

where F is an estimated amount of friction between a plunger and a barrel, w is a trained weight for the corresponding input generated by the ML model, and x is an input to the ML model (e.g., programmed flow rate or syringe extension). Likewise, a single-input ML model may be expressed as:

F=β₀+β₁Q+ . . . +βₙQⁿ

where F is, for example, an estimated mean amount of friction between a plunger and a barrel over all extensions, β represents the set of trained values, and Q represents the set of input values such as flow rate, syringe extension, and the like. In some implementations, the ML model may require iterative feature-selection in order to optimize for accuracy while avoiding over-fitting. As yet another example, a third, optimized model may be expressed as follows:

D~β₀+β₁E+β₂Q+β₃EQ+β₄E²+β₅E²Q+β₆E³+β₇E⁴+β₈E⁵

where D is drag (e.g., expected friction), β represents the set of trained weight coefficients, E is syringe extension, and Q is flow rate. Machine learning such as regression modeling, may identify an optimal set of features such as extension and flow rate along with corresponding weights. The optimal set may be those values and inputs that provide drag most closely matching observed drag in an actual syringe.

In some implementations, the syringe pump may be configured to select a specialized ML model, such as one of the aforenoted ML models trained using selective training data (e.g., controlling for an aspect of the syringe). For example, the syringe pump can be configured to detect or receive an identifier of the syringe. Then, based on the syringe identifier, the syringe pump may determine a quality of the syringe-such as a diameter of the syringe (e.g., an inner diameter or an outer diameter), a capacity of the syringe, a manufacturer of the syringe, or a model of the syringe. Based on the determined syringe quality, the syringe pump may then select an ML model from a plurality of ML models (e.g., stored on the syringe pump), where the training data used in training the ML model controlled for the aforenoted syringe quality. Moreover, in some implementations, the syringe pump is configured to report the syringe quality to a database. The data may be used, for example, to determine which syringe qualities are most common and further refine and train ML models accordingly.

As an example, after determining that the syringe has an inner diameter of 75 mm, the syringe pump may select a specialized ML model trained using data only from syringes with inner diameters of 75 mm. In some implementations, if the syringe pump is unable to locate such an ML model, the syringe pump may search for other ML models trained using closest fit data, and/or other controlled data corresponding to other qualities of the syringe in the syringe pump (e.g., having the same capacity of the syringe, manufactured by the same company as the syringe).

Moreover, in some implementations, the training data provided to an ML model is collected without using a syringe pump (e.g., using a material tester). In this manner, the ML model can be trained to be pump-agnostic (e.g., able to provide an estimated amount of friction for a syringe regardless of the syringe pump into which the syringe is loaded). In some implementations, a material tester is used to collect training data for the ML model because a material tester may be able to more accurately drive the plunger and more accurately measure the amount of friction experienced by the plunger. The material tester may be configured, for instance, to apply a force to the plunger and/or detect an amount of force applied by the plunger to a fluid within the syringe and provide measurements indexed according to the syringe extension. Furthermore, in some implementations, the ML model comprises a regression model that includes at least one trained weighting coefficient for the detected extension or the programmed flow rate.

It is noted that a multi-dimensional database or a lookup table may be used as an alternative to or in addition to an ML model. The same parameters may be used as inputs (e.g., as indices) to the database or the lookup table as those discussed above with respect to ML models (e.g., syringe extension, flow rate). Likewise, the same outputs (e.g., friction between the plunger and the barrel) may be obtained from the database or the lookup table.

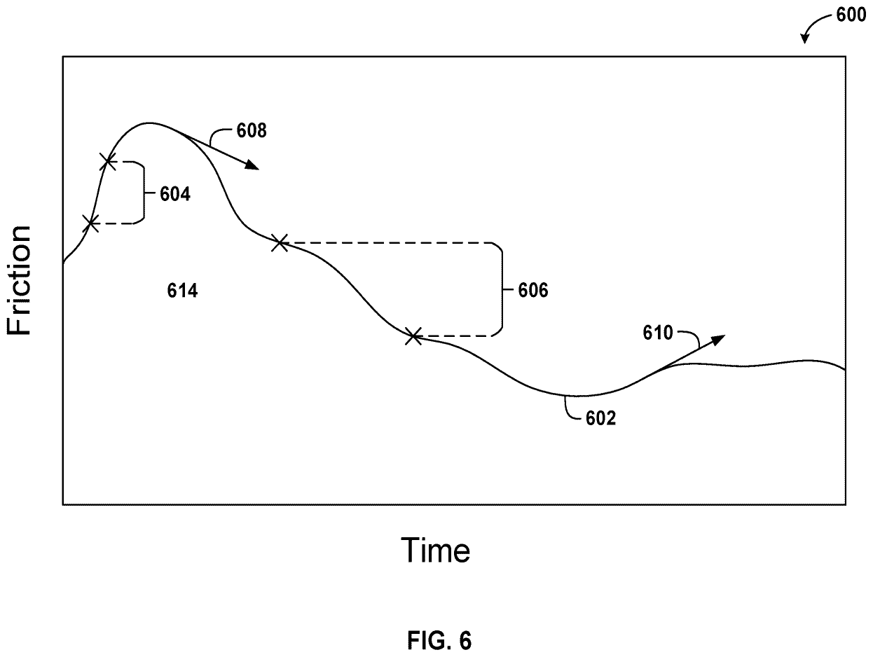

FIG. 6 is an example line graph 600 that includes a friction-estimation curve 602, according to various aspects of the subject technology. As depicted, the amount of friction 606 between a plunger (e.g., plunger 316 of FIG. 3, syringe plunger 406 of FIGS. 4A-4C, or plunger 508 of FIG. 5) and a barrel (e.g., barrel 308 of FIG. 3, syringe barrel 412 of FIGS. 4A-4C, or barrel 510 of FIG. 5) of a syringe (e.g., syringe 304 of FIG. 3, syringe 402 of FIGS. 4A-4C, or syringe 506 of FIG. 5) may vary over the course of an infusion therapy performed by a syringe pump (e.g., syringe pump 132 of FIG. 1, syringe pump module 302 of FIG. 3, or syringe pump 400 of FIGS. 4A-4C). The depicted curve 602 may be representative of real time infusion conditions.

As discussed with respect to FIG. 5, the amount of friction 602 resisting free movement of the plunger against the barrel (see force 503 of FIG. 5) can cause the amount of force between the plunger and the drive head (see force 501 of FIG. 5) to differ from the amount of force between the plunger and the fluid within the syringe (see force 502 of FIG. 5).

This difference can lead to errant occlusion alarms and unintended shifts in the flow rate of a syringe pump. For example, if the amount of force detected at the drive head is high due to friction, the infusion pump may incorrectly assume that the pressure in the syringe is also high and trigger an occlusion alarm-when in fact the syringe pressure is not high, and no occlusion exists. Similarly, changes in the friction between the plunger and the barrel may translate into unintended changes in the flow rate of the syringe pump. High friction, for instance, may slow the flow rate of the syringe pump. And friction variance may disrupt flow rate continuity. Accordingly, in order to avoid improper occlusion alarms and to maintain flow continuity, in some implementations, the syringe pump is configured to adjust (e.g., based on the estimated amount of friction) its own operation, such as an occlusion-detection operation or the operating speed of its drive head (e.g., drive head 324 of FIG. 3, drive head 408 of FIGS. 4A-4C, or drive head 504 of FIG. 5) to account for friction between the plunger and the barrel of the syringe.

Adjustments to a given parameter may be made responsive to various aspects of the estimated amount of friction 602. In some implementations, the adjustment can be made based on a difference between friction estimates taken at two different times. For example, if a friction estimate is greater than an earlier estimate (see bracket 604), the syringe pump may increase a pressure threshold used in occlusion detection. This may prevent an occlusion alarm from triggering prematurely. As another example, if a friction estimate is less than an earlier estimate (see bracket 606), the syringe pump may decrease the operating speed of the drive head motor. This may cause the syringe pump to operate at a flow rate closer to a programmed flow rate, and it may also avoid unintended changes in the flow rate of the syringe pump.

In making these adjustments, the degree of the adjustment may be based on the difference between the two friction estimates. For example, the difference between the friction estimates associated with bracket 604 is less than the difference between the estimates associated with bracket 606. Accordingly, the syringe pump may make a more significant adjustment (e.g., to the occlusion threshold or the operating speed) in response to the estimates associated with the second bracket 606. In either instance, if the syringe pump determines that the difference between two friction estimates is insignificant, then the syringe pump may not make any adjustment to its operation. Furthermore, the degree of the adjustment may also or alternatively be based on providing parameters (e.g., one or more friction estimates) to an ML model.

Additionally, in some implementations, the syringe pump adjustments may be made based on a slope of the friction curve 602. For example, if the slope is negative (see tangential arrow 608), the syringe pump may decrease a pressure threshold used in occlusion detection. As another example, if the slope of the friction curve 602 is positive (see tangential arrow 610), the syringe pump may increase the operating speed of the drive head motor. As with the adjustments discussed above with respect to brackets 604 and 606, the degree of the adjustment made by the syringe pump can be based on the magnitude of the slope of the friction curve 602—as opposed to simply considering whether the slope is positive or negative.

FIG. 7 depicts an example process 700 for improving occlusion detection or flow continuity of a syringe pump, according to various aspects of the subject technology. For explanatory purposes, the present disclosure describes the blocks of example process 700 with reference to FIGS. 1-6, including the components and/or processes described therein. One or more of the blocks of process 700 may be implemented by one or more of the computing devices described herein, such as the infusion device 100 of FIG. 1, the patient care device 202 of FIG. 2, the syringe pump module 302 of FIG. 3, and/or the syringe pump 400 of FIG. 4.

In some implementations, one or more of the blocks may be implemented based on one or more ML algorithms. In some implementations, one or more of the blocks may be implemented apart from other blocks, and by one or more different processors or devices. Further, for explanatory purposes, the blocks of the process 700 are described as occurring in serial, or linearly. However, multiple blocks of the process 700 may occur in parallel. Additionally, the blocks of the process 700 need not be performed in the order shown and one or more of the blocks of the process 700 need not be performed.

In the depicted example, a processor (e.g., CPU 218 of FIG. 2) receives a programmed flow rate for a syringe pump (e.g., infusion device 100 of FIG. 1, patient care device 202 of FIG. 2, syringe pump module 302 of FIG. 3, or syringe pump 400 of FIG. 4) (702). The programmed flow rate can be received prior to starting an infusion therapy via the syringe pump. However, the programmed flow rate may also be received in the middle of the infusion therapy (e.g., titration programming). Additionally, the programmed flow rate may correspond to an operating speed of the drive head or a motor or other element for advancing the syringe plunger.

The processor also detects an extension of a syringe received within a receptacle (e.g., cradle 306 of FIG. 3) of the syringe pump (704). The syringe includes a plunger (e.g., plunger 316 of FIG. 3, syringe plunger 406 of FIGS. 4A-4C, or plunger 508 of FIG. 5) and a barrel (e.g., barrel 308 of FIG. 3, syringe barrel 412 of FIGS. 4A-4C, or barrel 510 of FIG. 5). As noted above with respect to FIG. 4C (see distance 424 of FIG. 4C), the processor may detect the syringe extension based on the programmed flow rate (e.g., and a corresponding motor speed or velocity of the drive head) and an amount of time for which an infusion therapy administered by the syringe pump has been running. Alternatively, or additionally, the processor may detect the syringe extension based on extension sensors (e.g., optical sensors) affixed to the syringe pump and configured to locate a position of the drive head or a position of the syringe (e.g., of the flange of the plunger).

Additionally, the processor estimates (e.g., based on the programmed flow rate and the detected extension) an amount of friction between the plunger and the barrel (706). As discussed with respect to FIG. 5, in some implementations, estimating the amount of friction includes providing parameters (e.g., programmed flow rate or detected extension) to a model (e.g., an ML model) and obtaining the estimated amount of friction from the model. The model may be trained using training data regarding respective amounts of friction at respective extensions of a plurality of syringes operated at a plurality of flow rates. Moreover, in some implementations, estimating the amount of friction includes indexing into a lookup table (e.g., a multi-dimensional database) and obtaining an estimate from the lookup table.

In some implementations, the processor receives an identifier of the syringe. Based on the syringe identifier, the processor then determines a quality of the syringe. For example, the syringe quality may include a diameter of the syringe (e.g., inner diameter), a capacity of the syringe, a manufacturer of the syringe, or a model of the syringe. After determining the syringe quality, the processor selects an ML model from a plurality of ML models. The selected ML model may be a specialized ML model, for instance, trained using data collected from syringes each having a same quality as the syringe quality. For example, if the syringe in the syringe pump has a capacity of 50 milliliters, then the selected ML model may have been trained only using data from syringes each having 50 milliliter capacity. Once the process has selected the ML model, the processor then provides parameters to the ML model and receives the estimated friction from the ML model.

In some implementations, the training data used to train the ML model is collected using a material tester and without using a syringe pump. As noted above, a material tester may be able to produce more accurate testing data than a syringe pump. Further, using a material tester to collect training data means that the resulting ML model is pump-agnostic and can be used for a variety of syringe pumps, rather than just syringe pumps of a particular type or brand.

In some implementations, estimating the amount of friction between the plunger and the barrel is further based on a quality of the syringe. For example, the syringe quality could be a diameter of the syringe, a capacity of the syringe, a manufacturer of the syringe, or a model of the syringe. In this regard, the model may be trained with various quality parameters and the quality parameter of the syringe in the current infusion may be used (e.g., with other parameters) to obtain the estimated amount of friction. Accordingly, the processor may be configured to detect the diameter of the syringe via a diameter sensor of the syringe pump (or detect another syringe quality via another sensor configured for detecting the same). Alternatively, or additionally, the processor may be configured to receive an identifier of the syringe and determine the syringe quality based on the syringe identifier.

Further, the processor adjusts (e.g., based on the estimated amount of friction) the operation of the syringe pump (708). In some implementations, adjustment includes adjusting an occlusion-detection operation of the syringe pump (708A). In some implementations, the processor adjusts an operation speed of a drive head of the syringe pump (e.g., drive head 324 of FIG. 3, drive head 408 of FIGS. 4A-4C, or drive head 504 of FIG. 5) (708B).

In some implementations, the syringe pump includes an occlusion alarm and a force sensor affixed to the drive head and configured to detect an amount of force applied by the plunger to the drive head. Accordingly, adjusting the occlusion-detection operation of the syringe pump may include measuring, via the force sensor, the amount of force applied by the plunger to the drive head. Adjusting the occlusion-detection operation may also include determining, based on a difference between the estimated amount of friction and the measured amount of force, an amount of pressure in the syringe. Further, adjusting the occlusion-detection operation may include determining that the amount of pressure satisfies a pressure threshold and triggering the occlusion alarm responsive to determining that the amount of pressure in the syringe satisfies the pressure threshold.

In some implementations, the syringe pump includes an occlusion alarm configured to trigger when an amount of pressure in the syringe satisfies a pressure threshold (e.g., 525 mmHg, 12 psi, etc.). Accordingly, the occlusion-detection operation of the syringe pump may include adjusting the pressure threshold based on the estimated amount of friction. In this manner, the processor may prevent the occlusion alarm from triggering improperly based on an unadjusted pressure threshold. The threshold adjustment, for example, may be based on a difference between two estimated amounts of friction (e.g., at two different times) (see brackets 604 and 606 of FIG. 6, representing differences between respective friction estimates). As another example, the processor may periodically estimate the friction between the plunger and the barrel and the adjustment may be based on a slope of a curve including the friction estimates (see tangential arrows 608 and 610 of FIG. 6, representing slopes of friction curve 602).

In some implementations, adjusting the operating speed of the drive head is further based on a slope of the estimated amount of friction. Accordingly, adjusting the operating speed may include increasing or decreasing the operating speed responsive to determining that the slope of the estimated amount of friction is, respectively, positive or negative. The degree to which the operating speed is adjusted may be based on the magnitude of the slope. Further, the processor may be configured to periodically detect the extension of the syringe, periodically estimate the amount of friction between the plunger and the barrel (e.g., based on the programmed flow rate and/or the detected extension), and determine the slope of the estimated amount of friction based on periodically estimating the amount of friction. Alternatively, or additionally, the processor may adjust the operating speed based on a difference between two different friction estimates, as discussed above with respect to pressure threshold adjustments.

In some implementations, adjusting the occlusion-detection operation of the syringe pump, or the operating speed of the drive head, is further based on a viscosity of a fluid contained within the syringe. The fluid viscosity may determine, for instance, the amount of pressure necessary to pump the fluid out of the syringe at the programmed flow rate. Adjusting the pressure threshold or the operating speed may thus be necessary to avoid improper occlusion alarms and/or to ensure flow rate accuracy. Accordingly, the processor may be configured to receive an indicator of a type of the fluid, and determine the viscosity of the fluid based on the fluid type indicator.

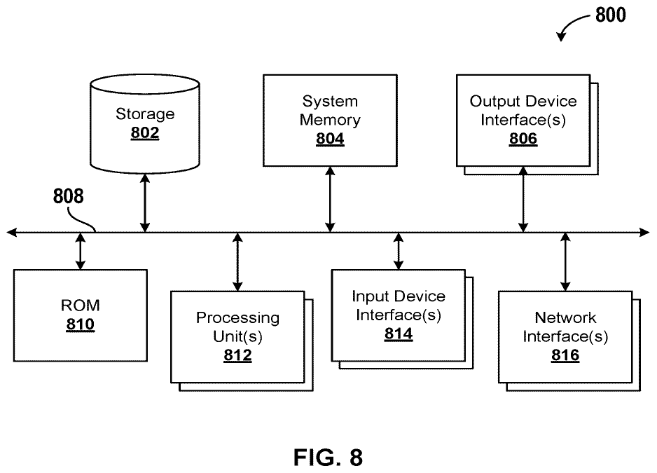

FIG. 8 is a conceptual diagram illustrating an example electronic system 800 for improving occlusion detection or flow continuity of a syringe pump, according to various aspects of the subject technology. Electronic system 800 may be implemented by a computing device for execution of software associated with portions or steps of process 700 of FIG. 7, or components and methods provided by FIGS. 1-6. In this regard, the electronic system 800 may include the infusion device 100 of FIG. 1, the patient care device 202 of FIG. 2, the syringe pump module 302 of FIG. 3, and/or the syringe pump 400 of FIG. 4.

The electronic system 800 may also include a specifically-configured personal computer or a mobile device for infusion such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

Additionally, the electronic system 800 may include various types of computer-readable media and interfaces for various other types of computer-readable media. In the depicted example, electronic system 800 includes a bus 808, a processing unit(s) 812, a system memory 804, a read-only memory (ROM) 810, a permanent storage device 802, an input device interface(s) 814, an output device interface(s) 806, and a network interface(s) 816. In some implementations, electronic system 800 may include or be integrated with other computing devices or circuitry for operation of the various components and methods previously described.

Bus 808 collectively represents system, peripheral, and chipset buses that communicatively connect the numerous internal devices of electronic system 800. For instance, bus 808 communicatively connects processing unit(s) 812 with ROM 810, the system memory 804, and permanent storage device 802. From these various memory units, processing unit(s) 812 retrieves instructions to execute and data to process in order to execute the processes of the subject disclosure. Processing unit(s) 812 can be a single processor or a multi-core processor in different implementations.

ROM 810 stores static data and instructions that are needed by processing unit(s) 812 and other modules of the electronic system. Permanent storage device 802, on the other hand, is a read-and-write memory device. This device is a non-volatile memory unit that stores instructions and data even when electronic system 800 is powered off. Some implementations of the subject disclosure use a mass-storage device (such as a magnetic or optical disk and its corresponding disk drive) as permanent storage device 802. Other implementations use a removable storage device (such as a floppy disk, flash drive, and its corresponding disk drive) as permanent storage device 802.

Like permanent storage device 802, system memory 804 is a read-and-write memory device. However, unlike storage device 802, system memory 804 is a volatile read-and-write memory, such as random-access memory (RAM). System memory 804 stores some of the instructions and data that the processor needs at runtime. In some implementations, the processes of the subject disclosure are stored in system memory 804, permanent storage device 802, and/or ROM 810. From these various memory units, processing unit(s) 812 retrieves instructions to execute and data to process, in order to execute the processes of some implementations.

Bus 808 also connects to input device interface(s) 814 and output device interface(s) 806. Input device interface(s) 814 enables the user to communicate information and select commands to the electronic system. Input devices used with input device interface(s) 814 include, for example, alphanumeric keyboards and pointing devices (also called “cursor control devices”). Output device interface(s) 806 enables, for example, the display of images generated by electronic system 800. Output devices used with output device interface(s) 806 include, for example, printers and display devices, such as cathode ray tubes (CRT) or liquid crystal displays (LCD). Some implementations include devices (e.g., touchscreens) that function as both input and output devices.

Furthermore, bus 808 also couples electronic system 800 to a network (not shown) through network interface(s) 816. Network interface(s) 816 may include, for example, a wireless access point (e.g., Bluetooth or Wi-Fi) or radio circuitry for connecting to a wireless access point. Network interface(s) 816 may also include hardware (e.g., ethernet hardware) for connecting the computer to a part of a network of computers such as a local area network (LAN), a wide area network (WAN), wireless LAN, an intranet, or a network of networks, such as the Internet. Components of electronic system 800 can be used in conjunction with the subject disclosure when specifically configured with one of more of the features described.

The functions described above can be implemented in computer software, firmware, or hardware. The techniques can be implemented using one or more computer program products. Programmable processors and computers can be included in or packaged as mobile devices. The processes and logic flows can be performed by one or more programmable processors and by programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field-programmable gate arrays (FPGAs). In some implementations, such integrated circuits execute instructions that are stored on the circuit itself.

As used in this specification and any claims of this application, the terms “computer,” “server,” “processor,” and “memory” all refer to electronic or other technological devices specifically configured with one or more of the features described above. These terms exclude people or groups of people. For the purposes of the specification, the terms display or displaying means displaying on an electronic device. As used in this specification and any claims of this application, the terms “computer-readable medium” and “computer-readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. For example, feedback provided to the user can be any form of sensory feedback (e.g., visual feedback, auditory feedback, tactile feedback), and input from the user can be received in forms such as acoustic, speech, gesture, or tactile input. In addition, a computer can interact with a user by sending documents to and receiving documents from a device that is used by the user (e.g., by sending web pages to a web browser on a user's client device in response to requests received from the web browser).

Implementations of the subject matter described in this specification can be implemented in a specifically configured computing system that includes a back end component (e.g., a data server), or that includes a specifically configured middleware component (e.g., an application server), or that includes a specifically configured front end component (e.g., a client computer having a graphical user interface or a Web browser through which a user can interact with an implementation of the subject matter described in this specification), or any combination of one or more such back end, middleware, or front end components. The components of the system can be interconnected by one or more forms or mediums of digital data communication, such as a communication network. Examples of communication networks include a LAN and a WAN, an inter-network (e.g., the Internet), and peer-to-peer networks (e.g., ad hoc peer-to-peer networks).

The computing system can include specifically configured clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art will appreciate that the various illustrative blocks, modules, elements, components, methods, and algorithms described herein may be implemented as electronic hardware, computer software, or a combination thereof. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.
Clause 1. A syringe pump comprising: a receptacle for receiving a syringe, the syringe comprising a plunger and a barrel; a drive head for advancing the plunger within the barrel; and a processor configured to: receive a programmed flow rate; detect an extension of the syringe, the extension comprising a distance between a starting position of the plunger within the barrel and a current position of the plunger within the barrel; estimate, based on the programmed flow rate and the detected extension, an amount of friction between the plunger and the barrel; and adjust, based on the estimated amount of friction, (i) an occlusion-detection operation of the syringe pump or (ii) an operating speed of the drive head.
Clause 2. The syringe pump of Clause 1, further comprising: an occlusion alarm; and a force sensor affixed to the drive head and configured to detect an amount of force applied by the plunger to the drive head; wherein adjusting the occlusion-detection operation of the syringe pump comprises: measuring, via the force sensor, the amount of force applied by the plunger to the drive head; determining, based on a difference between the estimated amount of friction and the measured amount of force, an amount of pressure in the syringe; determining that the amount of pressure satisfies a pressure threshold; and triggering the occlusion alarm responsive to determining that the amount of pressure in the syringe satisfies the pressure threshold.
Clause 3. The syringe pump of Clause 1 or Clause 2, further comprising an occlusion alarm configured to trigger when an amount of pressure in the syringe satisfies a pressure threshold, wherein adjusting the occlusion-detection operation of the syringe pump comprises adjusting the pressure threshold based on the estimated amount of friction.
Clause 4. The syringe pump of Clause 3, wherein: the estimated amount of friction comprises an estimated amount of friction between the plunger and the barrel at a first time; the processor is further configured to (i) detect another extension of the syringe, the other extension comprising a distance between the starting position of the plunger within the barrel and a position of the plunger within the barrel at a second time subsequent to the first time and (ii) estimate, based on the programmed flow rate and the other detected extension, another amount of friction between the plunger and the barrel; and adjusting the pressure threshold is based on a difference between the estimated amount of friction and the other estimated amount of friction.
Clause 5. The syringe pump of any one of Clauses 1 through 4, wherein adjusting the operating speed of the drive head is further based on a slope of the estimated amount of friction and comprises: increasing the operating speed responsive to determining that the slope of the estimated amount of friction is positive or decreasing the operating speed responsive to determining that the slope of the estimated amount of friction is negative; wherein the processor is further configured to (i) periodically detect the extension of the syringe, (ii) periodically estimate, based on the programmed flow rate and the detected extension, the amount of friction between the plunger and the barrel, and (iii) determine the slope of the estimated amount of friction based on periodically estimating the amount of friction.
Clause 6. The syringe pump of any one of Clauses 1 through 5, wherein: adjusting the occlusion-detection operation of the syringe pump, or the operating speed of the drive head, is further based on a viscosity of a fluid contained within the syringe; and the processor is further configured to (i) receive an indicator of a type of the fluid, and (ii) determine the viscosity of the fluid based on the fluid type indicator.
Clause 7. The syringe pump of any one of Clauses 1 through 6, wherein: estimating the amount of friction between the plunger and the barrel is further based on a quality of the syringe, the syringe quality comprising a diameter of the syringe, a capacity of the syringe, a manufacturer of the syringe, or a model of the syringe; and the processor is further configured to (i) detect the diameter of the syringe via a diameter sensor of the syringe pump, or (ii) receive an identifier of the syringe and determine the syringe quality based on the syringe identifier.
Clause 8. The syringe pump of any one of Clauses 1 through 7, wherein estimating the amount of friction between the plunger and the barrel comprises: providing the programmed flow rate and the detected extension of the syringe to a machine learning (ML) model trained using training data regarding respective amounts of friction at respective extensions of a plurality of syringes operated at a plurality of flow rates; and receiving the estimated amount of friction from the ML model.
Clause 9. The syringe pump of Clause 8, wherein: the processor is further configured to (i) receive an identifier of the syringe, (ii) determine a quality of the syringe based on the syringe identifier, the syringe quality comprising a diameter of the syringe, a capacity of the syringe, a manufacturer of the syringe, or a model of the syringe, and (iii) select, based on the syringe quality, the ML model from a plurality of ML models; and each of the plurality of syringes has the syringe quality.
Clause 10. The syringe pump of Clause 8 or Clause 9, wherein the ML model comprises a regression model that includes at least one trained weighting coefficient for the detected extension or the programmed flow rate.
Clause 11. A computer-implemented method for improving occlusion detection or flow continuity of a syringe pump, comprising: receiving a programmed flow rate for a syringe pump, the syringe pump comprising (i) a receptacle for receiving a syringe, the syringe comprising a plunger and a barrel, and (ii) a drive head for advancing the plunger within the barrel; detecting an extension of the syringe, the extension comprising a distance between a starting position of the plunger within the barrel and a current position of the plunger within the barrel; estimating, based on the programmed flow rate and the detected extension, an amount of friction between the plunger and the barrel; and adjusting, based on the estimated amount of friction, (i) an occlusion-detection operation of the syringe pump or (ii) an operating speed of the drive head.
Clause 12. The computer-implemented method of Clause 11, wherein adjusting the occlusion-detection operation of the syringe pump comprises: measuring, via a force sensor of the syringe pump, an amount of force applied by the plunger to the drive head; determining, based on a difference between the estimated amount of friction and the measured amount of force, an amount of pressure in the syringe; determining that the amount of pressure satisfies a pressure threshold; and triggering an occlusion alarm of the syringe pump responsive to determining that the amount of pressure in the syringe satisfies the pressure threshold.
Clause 13. The computer-implemented method of Clause 11 or Clause 12, wherein: the syringe pump further comprises an occlusion alarm configured to trigger when an amount of pressure in the syringe satisfies a pressure threshold; and adjusting the occlusion-detection operation of the syringe pump comprises adjusting the pressure threshold based on the estimated amount of friction.
Clause 14. The computer-implemented method of Clause 13, further comprising: detecting another extension of the syringe, the other extension comprising a distance between the starting position of the plunger within the barrel and a position of the plunger within the barrel at a second time; and estimating, based on the programmed flow rate and the other detected extension, another amount of friction between the plunger and the barrel; wherein the estimated amount of friction comprises an estimated amount of friction between the plunger and the barrel at a first time prior to the second time, and adjusting the pressure threshold is based on a difference between the estimated amount of friction and the other estimated amount of friction.
Clause 15. The computer-implemented method of any one of Clauses 11 through 14, further comprising: periodically detecting the extension of the syringe; periodically estimating, based on the programmed flow rate and the detected extension, the amount of friction between the plunger and the barrel; and determining a slope of the estimated amount of friction based on periodically estimating the amount of friction; wherein adjusting the operating speed of the drive head is further based on the slope of the estimated amount of friction and comprises (i) increasing the operating speed responsive to determining that the slope of the estimated amount of friction is positive or (ii) decreasing the operating speed responsive to determining that the slope of the estimated amount of friction is negative.
Clause 16. The computer-implemented method of any one of Clauses 11 through 15, further comprising: receiving an indicator of a type of a fluid contained within the syringe; and determining a viscosity of the fluid based on the fluid type indicator; wherein adjusting the occlusion-detection operation of the syringe pump, or the operating speed of the drive head, is further based on the fluid viscosity.
Clause 17. The computer-implemented method of any one of Clauses 11 through 16, further comprising: detecting a diameter of the syringe via a diameter sensor of the syringe pump; or receiving an identifier of the syringe and determining a quality of the syringe based on the syringe identifier, the syringe quality comprising a diameter of the syringe, a capacity of the syringe, a manufacturer of the syringe, or a model of the syringe; wherein estimating the amount of friction between the plunger and the barrel is further based on the syringe quality.
Clause 18. The computer-implemented method of any one of Clauses 11 through 17, wherein estimating the amount of friction between the plunger and the barrel comprises: providing the programmed flow rate and the detected extension of the syringe to a machine learning (ML) model trained using training data regarding respective amounts of friction at respective extensions of a plurality of syringes operated at a plurality of flow rates; and receiving the estimated amount of friction from the ML model.
Clause 19. The computer-implemented method of Clause 18, further comprising: receiving an identifier of the syringe; determining a quality of the syringe based on the syringe identifier, the syringe quality comprising a diameter of the syringe, a capacity of the syringe, a manufacturer of the syringe, or a model of the syringe; and selecting, based on the syringe quality, the ML model from a plurality of ML models; wherein each of the plurality of syringes has the quality of the syringe.
Clause 20. A non-transitory, computer-readable storage medium comprising instructions that, when executed by an electronic device, cause the electronic device to perform operations comprising: receiving a programmed flow rate for a syringe pump, the syringe pump comprising (i) a receptacle for receiving a syringe, the syringe comprising a plunger and a barrel, and (ii) a drive head for advancing the plunger within the barrel; detecting an extension of the syringe, the extension comprising a distance between a starting position of the plunger within the barrel and a current position of the plunger within the barrel; estimating, based on the programmed flow rate and the detected extension, an amount of friction between the plunger and the barrel; and adjusting, based on the estimated amount of friction, (i) an occlusion-detection operation of the syringe pump or (ii) an operating speed of the drive head.

### FURTHER CONSIDERATION

It is understood that the specific order or hierarchy of steps in the processes disclosed herein is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein, but is to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to”, “operable to”, and “programmed to” do not imply any particular tangible or intangible modification of a subject, but, rather, are intended to be used interchangeably. For example, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

The term automatic, as used herein, may include performance by a computer or machine without user intervention; for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such as an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some embodiments, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, an ML assessment model, or combinations thereof.

In any embodiment, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. A syringe pump comprising:
a receptacle for receiving a syringe, the syringe comprising a plunger and a barrel;
a drive head for advancing the plunger within the barrel; and
a processor configured to:
receive a programmed flow rate;
detect an extension of the syringe, the extension comprising a distance between a starting position of the plunger within the barrel and a current position of the plunger within the barrel;
estimate, based on the programmed flow rate and the detected extension, an amount of friction between the plunger and the barrel; and
adjust, based on the estimated amount of friction, (i) an occlusion-detection operation of the syringe pump or (ii) an operating speed of the drive head.

2. The syringe pump of claim 1, further comprising:
an occlusion alarm; and
a force sensor affixed to the drive head and configured to detect an amount of force applied by the plunger to the drive head;
wherein adjusting the occlusion-detection operation of the syringe pump comprises:
measuring, via the force sensor, the amount of force applied by the plunger to the drive head;
determining, based on a difference between the estimated amount of friction and the measured amount of force, an amount of pressure in the syringe;
determining that the amount of pressure satisfies a pressure threshold; and
triggering the occlusion alarm responsive to determining that the amount of pressure in the syringe satisfies the pressure threshold.

3. The syringe pump of claim 1, further comprising an occlusion alarm configured to trigger when an amount of pressure in the syringe satisfies a pressure threshold, wherein adjusting the occlusion-detection operation of the syringe pump comprises adjusting the pressure threshold based on the estimated amount of friction.

4. The syringe pump of claim 3, wherein:
the estimated amount of friction comprises an estimated amount of friction between the plunger and the barrel at a first time;
the processor is further configured to (i) detect another extension of the syringe, the other extension comprising a distance between the starting position of the plunger within the barrel and a position of the plunger within the barrel at a second time subsequent to the first time and (ii) estimate, based on the programmed flow rate and the other detected extension, another amount of friction between the plunger and the barrel; and
adjusting the pressure threshold is based on a difference between the estimated amount of friction and the other estimated amount of friction.

5. The syringe pump of claim 1, wherein adjusting the operating speed of the drive head is further based on a slope of the estimated amount of friction and comprises:
increasing the operating speed responsive to determining that the slope of the estimated amount of friction is positive or decreasing the operating speed responsive to determining that the slope of the estimated amount of friction is negative;
wherein the processor is further configured to (i) periodically detect the extension of the syringe, (ii) periodically estimate, based on the programmed flow rate and the detected extension, the amount of friction between the plunger and the barrel, and (iii) determine the slope of the estimated amount of friction based on periodically estimating the amount of friction.

6. The syringe pump of claim 1, wherein:
adjusting the occlusion-detection operation of the syringe pump, or the operating speed of the drive head, is further based on a viscosity of a fluid contained within the syringe; and
the processor is further configured to (i) receive an indicator of a type of the fluid, and (ii) determine the viscosity of the fluid based on the fluid type indicator.

7. The syringe pump of claim 1, wherein:
estimating the amount of friction between the plunger and the barrel is further based on a quality of the syringe, the syringe quality comprising a diameter of the syringe, a capacity of the syringe, a manufacturer of the syringe, or a model of the syringe; and
the processor is further configured to (i) detect the diameter of the syringe via a diameter sensor of the syringe pump, or (ii) receive an identifier of the syringe and determine the syringe quality based on the syringe identifier.

8. The syringe pump of claim 1, wherein estimating the amount of friction between the plunger and the barrel comprises:
providing the programmed flow rate and the detected extension of the syringe to a machine learning (ML) model trained using training data regarding respective amounts of friction at respective extensions of a plurality of syringes operated at a plurality of flow rates; and
receiving the estimated amount of friction from the ML model.

9. The syringe pump of claim 8, wherein:
the processor is further configured to (i) receive an identifier of the syringe, (ii) determine a quality of the syringe based on the syringe identifier, the syringe quality comprising a diameter of the syringe, a capacity of the syringe, a manufacturer of the syringe, or a model of the syringe, and (iii) select, based on the syringe quality, the ML model from a plurality of ML models; and
each of the plurality of syringes has the syringe quality.

10. The syringe pump of claim 8, wherein the ML model comprises a regression model that includes at least one trained weighting coefficient for the detected extension or the programmed flow rate.

11. A computer-implemented method for improving occlusion detection or flow continuity of a syringe pump, comprising:
receiving a programmed flow rate for a syringe pump, the syringe pump comprising (i) a receptacle for receiving a syringe, the syringe comprising a plunger and a barrel, and (ii) a drive head for advancing the plunger within the barrel;
detecting an extension of the syringe, the extension comprising a distance between a starting position of the plunger within the barrel and a current position of the plunger within the barrel;
estimating, based on the programmed flow rate and the detected extension, an amount of friction between the plunger and the barrel; and
adjusting, based on the estimated amount of friction, (i) an occlusion-detection operation of the syringe pump or (ii) an operating speed of the drive head.

12. The computer-implemented method of claim 11, wherein adjusting the occlusion-detection operation of the syringe pump comprises:
measuring, via a force sensor of the syringe pump, an amount of force applied by the plunger to the drive head;
determining, based on a difference between the estimated amount of friction and the measured amount of force, an amount of pressure in the syringe;
determining that the amount of pressure satisfies a pressure threshold; and
triggering an occlusion alarm of the syringe pump responsive to determining that the amount of pressure in the syringe satisfies the pressure threshold.

13. The computer-implemented method of claim 11, wherein:
the syringe pump further comprises an occlusion alarm configured to trigger when an amount of pressure in the syringe satisfies a pressure threshold; and
adjusting the occlusion-detection operation of the syringe pump comprises adjusting the pressure threshold based on the estimated amount of friction.

14. The computer-implemented method of claim 13, further comprising:
detecting another extension of the syringe, the other extension comprising a distance between the starting position of the plunger within the barrel and a position of the plunger within the barrel at a second time; and
estimating, based on the programmed flow rate and the other detected extension, another amount of friction between the plunger and the barrel;
wherein the estimated amount of friction comprises an estimated amount of friction between the plunger and the barrel at a first time prior to the second time, and adjusting the pressure threshold is based on a difference between the estimated amount of friction and the other estimated amount of friction.

15. The computer-implemented method of claim 11, further comprising:
periodically detecting the extension of the syringe;
periodically estimating, based on the programmed flow rate and the detected extension, the amount of friction between the plunger and the barrel; and
determining a slope of the estimated amount of friction based on periodically estimating the amount of friction;
wherein adjusting the operating speed of the drive head is further based on the slope of the estimated amount of friction and comprises (i) increasing the operating speed responsive to determining that the slope of the estimated amount of friction is positive or (ii) decreasing the operating speed responsive to determining that the slope of the estimated amount of friction is negative.

16. The computer-implemented method of claim 11, further comprising:
receiving an indicator of a type of a fluid contained within the syringe; and
determining a viscosity of the fluid based on the fluid type indicator;
wherein adjusting the occlusion-detection operation of the syringe pump, or the operating speed of the drive head, is further based on the fluid viscosity.

17. The computer-implemented method of claim 11, further comprising:
detecting a diameter of the syringe via a diameter sensor of the syringe pump; or
receiving an identifier of the syringe and determining a quality of the syringe based on the syringe identifier, the syringe quality comprising a diameter of the syringe, a capacity of the syringe, a manufacturer of the syringe, or a model of the syringe;
wherein estimating the amount of friction between the plunger and the barrel is further based on the syringe quality.

18. The computer-implemented method of claim 11, wherein estimating the amount of friction between the plunger and the barrel comprises:
providing the programmed flow rate and the detected extension of the syringe to a machine learning (ML) model trained using training data regarding respective amounts of friction at respective extensions of a plurality of syringes operated at a plurality of flow rates; and
receiving the estimated amount of friction from the ML model.

19. The computer-implemented method of claim 18, further comprising:
receiving an identifier of the syringe;
determining a quality of the syringe based on the syringe identifier, the syringe quality comprising a diameter of the syringe, a capacity of the syringe, a manufacturer of the syringe, or a model of the syringe; and
selecting, based on the syringe quality, the ML model from a plurality of ML models;
wherein each of the plurality of syringes has the quality of the syringe.

20. A non-transitory, computer-readable storage medium comprising instructions that, when executed by an electronic device, cause the electronic device to perform operations comprising:
receiving a programmed flow rate for a syringe pump, the syringe pump comprising (i) a receptacle for receiving a syringe, the syringe comprising a plunger and a barrel, and (ii) a drive head for advancing the plunger within the barrel;
detecting an extension of the syringe, the extension comprising a distance between a starting position of the plunger within the barrel and a current position of the plunger within the barrel;
estimating, based on the programmed flow rate and the detected extension, an amount of friction between the plunger and the barrel; and
adjusting, based on the estimated amount of friction, (i) an occlusion-detection operation of the syringe pump or (ii) an operating speed of the drive head.
